(19) United States
(12) Patent Application Publication
(10) Pub. No.: US 2005/0096456 A1
(43) Pub. Date: May 5, 2005

(54) Compounds comprising pseudo-amino acids

(76) Inventor: Jason Richard Betley, Essex (GB)

Correspondence Address: HELLER EHRMAN WHITE & MCAULIFFE LLP, 1717 RHODE ISLAND AVE, NW, WASHINGTON, DC, 20036-3001, US

(21) Appl. No.: 10/508,854

(22) PCT Filed: Mar. 25, 2003
(86) PCT No.: PCT/GB03/01270

(30) Foreign Application Priority Data
Mar. 25, 2002 (GB) 0206981.3

(51) Int. Cl. C07K 7/08; C07K 7/06; C07K 5/06

(52) U.S. Cl. 530/324; 530/325; 530/326; 530/327; 530/328; 530/329; 530/330; 530/331; 562/557

(57) ABSTRACT

This invention relates to compounds, and in particular to polypeptides, their use and methods for their production. The polypeptides are produced from “pseudo-cystein” amino acids that facilitate the assembly of polypeptides by native chemical ligation. The invention provides a compound comprising a polypeptide, the polypeptide having at a C-terminal end a pseudo amino acid, the pseudo amino acid having a side chain containing a 1-amino-2-thiol moiety. For example the polypeptide comprises the structure: formula (I) wherein: R is a polypeptide chain; X is a linker.

This invention relates to compounds, and in particular to polypeptides, their use and methods for their production. These polypeptides are produced from “pseudo-cysteine” amino acids that facilitate the assembly of polypeptides by native chemical ligation.

### BACKGROUND

It is known that it is possible to ligate two peptidic or non-peptidic compounds together by a process known as native chemical ligation. The basic requirements for this coupling are the presence on participant A of a thioester moiety such as a benzyl or ethyl thioester, and the presence on participant B of an N-terminal cysteine. The coupling reaction commences when the thio-nucleophile initially attacks the thioester in a reversible fashion causing the thioalkyl group to leave. The thioester so formed then undergoes an intramolecular S→N acyl shift to irreversibly produce the ligated amide product A-B with a native cysteine at the junction (FIG. 1).

This process can be repeated if it is possible to unmask a further N-terminal cysteine at the N-terminal of A, in the product A-B. This can be achieved by enzymatic means or by classical protecting group chemistry. A further native chemical ligation coupling with a thioester-containing moiety E would therefore yield the product E-A-B. These methods have been used to generate a variety of proteins.

It is also known that cyclic peptides/proteins can be produced by incorporating both the thioester and amino terminal cysteine (N-terminal cysteine) moieties within the same molecule.

Further, it is possible to perform the ligation described above without removing all of the reaction participants from a solid phase.

A method for incorporating a thioester moiety into a participant where one does not exist is also known. This method relies on the reaction of a primary amine with thiolane-2,5-dione and subsequent alkylation of the thioacid formed with an appropriate alkyl bromide. This method requires a single primary amine to be present otherwise multiple thioesters may be coupled together.

Amino-acid monomer molecules are available with a protected functionality grafted onto another amino acid sidechain. For example, N-α-Fmoc-N-β(N-t-Boc-amino-oxyacetyl)-L-diaminopropionic acid (Novabiochem-San Diego, Calif.) incorporates a protected oxyamine onto an amino acid sidechain that is capable, after deprotection, of undergoing chemoselective ligation to carbonyl containing moieties. However, with reagents currently available it is not possible to incorporate a pseudo C-terminal cysteine into a polypeptide. A C-terminal cysteine is desirable to allow the linking of a molecule to a thioester (vide supra) without using an N-terminal cysteine, if for example, the N-terminus is required unmodified for biological activity.

Further, there is no method to enable the ligation of a C-terminal or other thioester with a pseudo C-terminal cysteine.

We have now produced reagents that enable the incorporation onto an amino acid sidechain at the C-terminal end of the peptide, a surrogate 1-amino-2-thiol that would be capable of performing a native chemical ligation reaction.

Further, we have disclosed a method that can be used to introduce a 1-amino-2-thiol group into the side chain of a lysine to enable the conjugate of a polypeptide containing such a residue to be ligated to a target compound using native chemical ligation methods.

### DETAIL OF THE INVENTION

The invention provides a compound comprising a polypeptide, the polypeptide having at a C-terminal end a pseudo amino acid, the pseudo amino acid having a side chain containing a 1-amino-2-thiol moiety.

Further it provides a compound having a polypeptide of the structure: 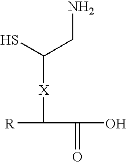
wherein:
R is a polypeptide chain;
X is a linker.

The linker may comprise, for example, methylene units i.e. CH₂ units or may include residues covalently linked by amide, ester, ether, thioether bonds or aryl groups, for example. Further, the linker may be (CH2)n, where n is 0 to 6, preferably 4.

The invention also provides a protected pseudo amino acid comprising the structure 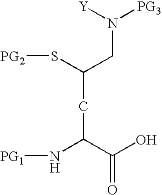
where the Y is optionally H or other suitable residue and PG1, PG2 and PG3 are different protecting groups.

The protecting groups may be FMOC, BOC or Trt, for example although other will be known to those skilled in the art.

One suitable protected amino acids is 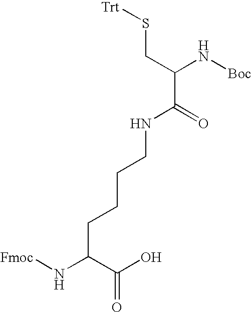
which may be produced as described in Example 2 and illustrated in FIG. 2.

WO 98/02454 relates to the derivatisation of soluble complement regulators by disulfide exchange reaction with pyridyl disulfide activated myristoyl/electrostatic switch peptides (MSWPs). These derivatives demonstrate improved complement inhibitory activity owing to their membrane localisation by the MSWP. The myristoyl group was designed to be minimally membrane insertive, while the lysine rich peptidic portion of MSWP-1 (Seq. ID No 1) was designed to interact with the negatively charged phospholipid headgroups present on the lipid bilayer. The concept is exemplified in WO 98/02454 by SCR1-3 of LHR-A of CR1, altered by a single C-terminal cysteine residue. After treatment with an appropriate reducing agent to ensure the presence of a free thiol on the terminal cysteine sidechain, the protein is reacted with MSWP-1 (N-(Myristoyl)-Gly-Ser-Ser-Lys-Ser-Pro-Ser-Lys-Lys-Lys-Lys-Lys-Lys-Pro-Gly-Asp-(S-2-Thiopyridyl)Cys-NH₂) (Seq ID No: 1) to yield a membrane binding derivative of SCR1-3 (FIG. 4). The resultant conjugate protein showed anti-haemolytic activity orders of magnitude greater than that displayed by the underivatised protein alone.

This chemistry relies on the existence within the protein participant of the reaction a single free thiol resulting in derivatisation at a single point only. In other circumstances it is desirable to link the class of peptide described to proteinaceous or non-proteinaceous molecules bearing a thioester. In the current design of the peptide, however, an N-terminal myristoyl function is required, thus disallowing the incorporation of an N-terminal cysteine to participate in the ligation reaction.

In order to achieve this, we describe an amino acid monomer capable of participating in standard Fmoc synthesis that would yield, on final deprotection a 1-amino-2-thiol at the c-terminus (FIG. 2 and Example 2). When incorporated into a polypeptide containing myristoyl switch type peptide (APT2278-Seq ID No: 2), it was indeed found to be capable of undergoing native chemical ligation to three widely differing thioester-containing molecules, a vitamin, a polypeptide, and an Impact™ expressed protein thioester.

Also disclosed is the incorporation of such a protected sidechain 1-amino-2-thiol into a peptide by the use of orthogonal protecting groups (Example 3).

Where the thioester-containing moiety is a protein, one method of formation of the ligated product of the invention is provided by the IMPACT system (New England Biolabs). Conjugates are afforded by the native chemical ligation of peptides containing an N-terminal cysteine, to proteins expressed in frame with a controllable intervening peptide sequence (CIVPS) and an affinity purification handle. The desired protein is cleaved from its affinity purification matrix by elution with a free thiol such as MESNA to yield a C-terminal thioester. The MESNA thioester is displaced by the thiol of the N-terminal cysteine before an S→N acyl shift installs the peptide bond in an irreversible fashion. Alternatively, the affinity matrix is incubated with the free peptide, displacing the desired protein from the matrix and ligating in a single step.

In the specific case where there is a 1-amino-2-thiol engineered onto a C-terminal sidechain and additionally there is an N-terminal cysteine, (eg FIG. 3 and Example 4) it is possible to undergo native chemical ligation on both ends of the molecule. If both 1-amino-2-thiols react with the same thioester it is possible to form a homodimer (FIG. 4, Example 5). If one 1-amino-2-thiol is reacted first and another after a subsequent purification, heterodimeric structures are possible (FIG. 5, Example 6).

It is also possible to incorporate more than one sidechain 1-amino-2-thiol into peptides utilising this technology to allow more than two moieties to be linked (FIG. 6 and Example 7). Different lengths and compositions of linker peptide between sidechain 1-amino-2-thiols can be used to afford differing spatial and physicochemical properties.

It is also possible to introduce other reactive groups for instance an amino-oxy function on the N-terminus to allow chemically orthogonal linking to other molecules after the completion of native chemical ligation.

Any of the components brought together with this enabling technology may be selected from the group consisting of:
A pharmacophore
A ligand
A small molecule
A purification handle/immunochemical tag (eg biotin)
A fluorescent moiety
A solubilising agent
A chelating ligand
A chelating ligand plus radioimaging agent
A therapeutic protein
An antibody or fragment thereof
A peptide
An enzyme

This list is not exhaustive. Other moieties may be linked in the fashion to be described and will be apparent to those skilled in the art.

### DRAWINGS

FIG. 1: Native Chemical Ligation schematic.

FIG. 2: Synthesis of a monomer to allow single-step incorporation of a protected sidechain 1-amino-2-thiol into a peptide by solid phase synthesis.

FIG. 3: Structure of dimerisation reagent APT3068.

FIG. 4: Preparation of a homodimer using the technology.

FIG. 5: Preparation of a heterodimer using the technology.

FIG. 6: Structure of trimerisation reagent APT3288.

FIG. 7: IMPACT system of preparing polypeptides

FIG. 8: Scheme 1 for preparing protected pseudo amino acids.

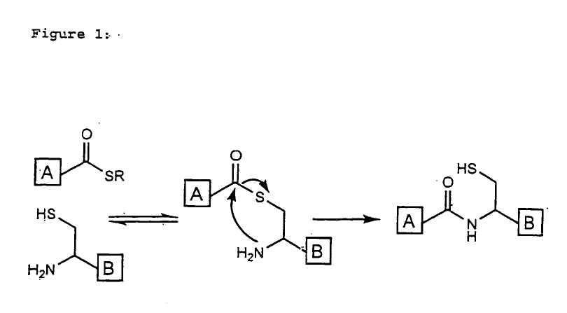

### EXAMPLES

### Example 1

### Synthesis of Disulfide Exchange MSWP1 (SEQ ID NO: 1)

The Peptide:

Myristoyl-Gly-Ser-Ser-Lys-Ser-Pro-Ser-Lys-Lys-Lys-Lys-Lys-Lys-Pro-Gly-Asp-Cys-(S-2-thiopyridyl)-NH₂ was synthesised according to the method described in WO 98/02454.

### Example 2

### Synthesis of Protected Lysine Sidechain 1-amino-2-thiol Amino Acid Monomer

N-α-Fmoc-N-ε-Boc-L-Lysine (5.14 g) was dissolved in 50/50 DCM/TFA (˜20 mL) and stirred for 30 minutes. The solvent was evaporated under reduced pressure and the residual yellow oil was dissolved in water and freeze-dried to remove remaining TFA and furnish N-α-Fmoc-L-Lysine.

A solution of Boc-Cys(Trt)-OH (4.66 g) and N-hydroxysuccinimide (1.15 g) in 1,4-dioxane (20 mL) and DCC (dcyclohexylcarbodiimide) (2.06 g) were stirred overnight. The urea derivative, which precipitated, was filtered off and washed with DCM. Solvent was evaporated and the resulting oil dissolved in ethyl acetate, then washed with sat. sodium carbonate solution then water. After drying over anhydrous sodium sulphate, the solvent was removed under reduced pressure to yield Boc-Cys(Trt)-N-hydroxy succinimide ester.

N-α-Fmoc-L-Lysine above was dissolved in DCM (20 mL) and the Boc-Cys(Trt)-N-hydroxysuccinimide ester above dissolved in DMF (30 mL) and DCM (10 mL). The solutions were mixed and DIPEA added until pH 7. The mixture was stirred overnight at ambient temperature. After this time, half the solvent was removed under reduced pressure and ethyl acetate-(200 mL) added. The organic mixture was washed with water (100 mL), sodium bicarbonate (200 mL), and water (100 mL). The organic layer was dried over anhydrous sodium sulphate and evaporated to yield a cream coloured foam. The product was purified using silica gel chromatography using 5:1 ethyl acetate/petroleum ether 40-60 to afford the product as a pale cream foam.

### Example 3

### Synthesis of Native Chemical Ligation Peptide MSWP2278 (SEQ ID NO: 2)

The Peptide:

Myristoyl-Gly-Ser-Ser-Lys-Ser-Pro-Ser-Lys-Lys-Asp-Asp-Lys-Lys-Pro-Gly-Asp-(E-amino-cysteinyl carboxamide)-Lys-NH₂ was prepared by solid phase synthesis using Boc synthesis on MBHA resin (Nova). Coupling reactions were carried out using appropriately protected Boc amino acid monomers (Nova) activated with TBTU aided with HOBT (Alexis Bio. Co.) with ninhydrin monitoring after each extension. The first amino acid was installed as α-amino-Boc, ε-amino-Fmoc lysine, and the Fmoc protection then removed with 20% piperidine. α-Amino-Fmoc, S-methoxybenzyl cysteine was then coupled to the ε-amino group before the remainder of the synthesis was carried out using appropriately protected Boc monomers. Cleavage from the resin and cleavage of the sidechain protecting groups was accomplished with high HF conditions using p-cresol and p-thiocresol as scavengers at 0° C. over 1 h.

The crude peptide was desalted via gel filtration (Sephadex G10, 0.1% TFA in water) before purification by preparative C18 Vydac) high performance liquid chromatography (HPLC) using 0.1% trifluoroacetic acid and 0.1% trifluoroacetic acid/90% acetonitrile as gradient components. After lyophilisation, the peptide was a white amorphous powder, soluble to at least 10 mg/mL in both dimethylsulfoxide and water. Fast atom bombardment spectrometry gave a main peak at 2101 amu corresponding to the molecular ion of the peptide.

### Example 4

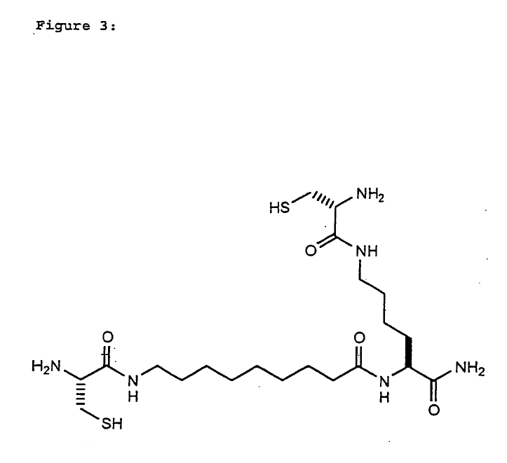

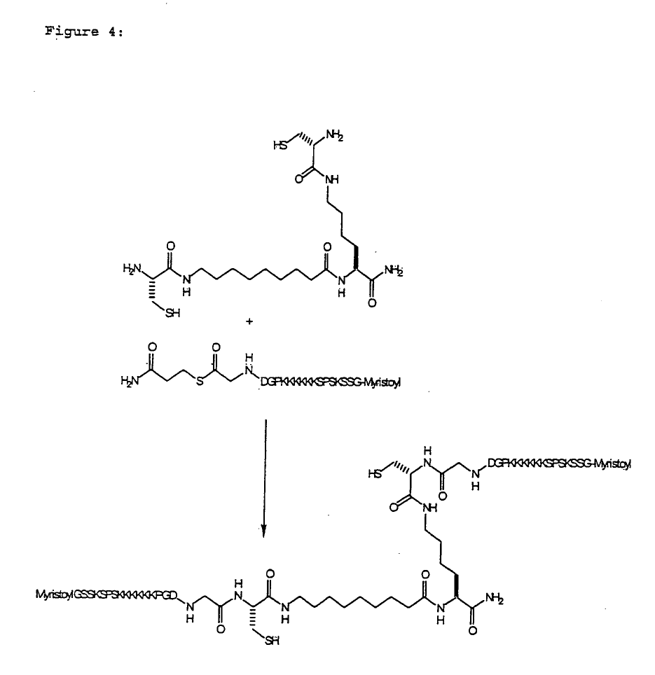

### Synthesis of Dimerisation Reagent (APT3068) (FIG. 3)

Standard Fmoc solid phase synthesis was utilised on Rink amide resin. Monomer was coupled first followed by Fmoc-9-aminononanoic acid followed by Boc-Cys(Trt)-OH. After cleavage from the resin and removal of all protecting groups using TFA/EDT/TIPS (95/5/5) the crude linker was ether precipitated and purified by preparative C18-reverse phase HPLC. ESMS 507.2 (M+H).

### Example 5

### Formation of a Homodimer Using Native Chemical Ligation

A solution of APT3068 in water (37.5 mM, 21.3 uL) was added to a solution of TCEP (tris-2-carboxyethyl phosphine) (1 equiv.) in HEPES buffer (pH 8.5, 40 mM, 265 uL). A solution of APT2501 (10 mg/mL, 140 uL) was then added. The reaction mixture was left at ambient temperature overnight and then analysed by HPLC. The doubly ligated product eluted slightly later than APT2501 and was found to be a 2/1 ligation product by MALDI MS-4378. This product was purified by preparative HPLC and lyophilised.

### Example 6

### Formation of a Heterodimer Using Native Chemical Ligation

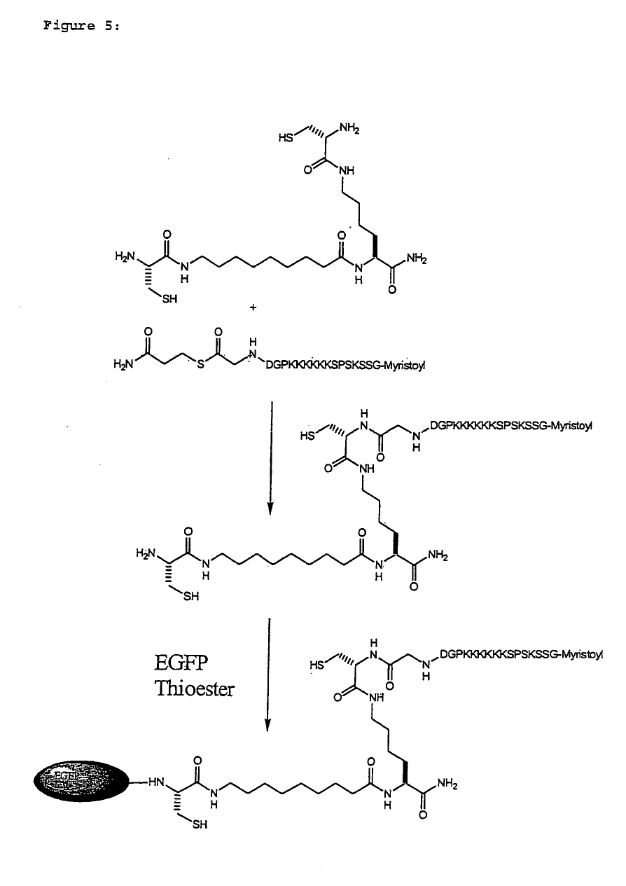

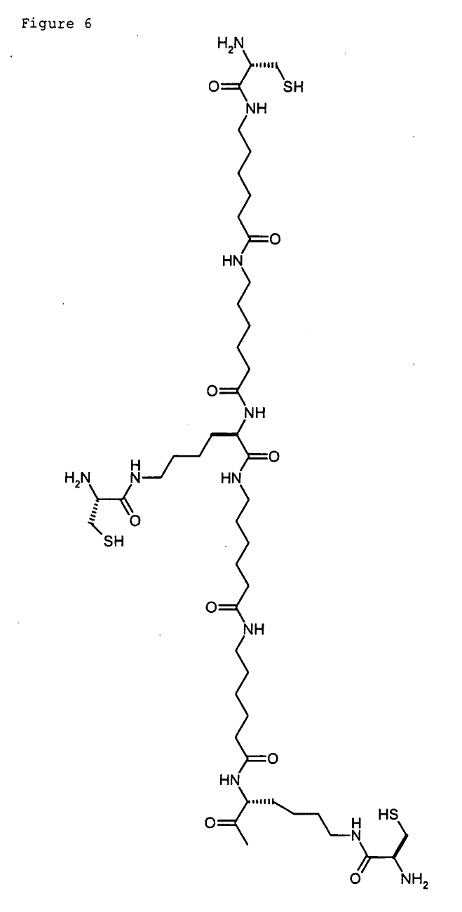

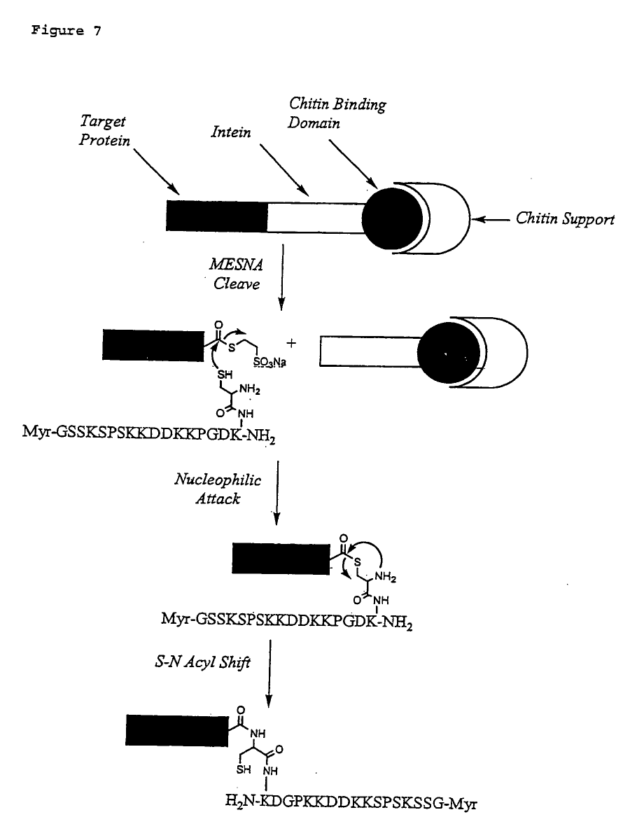

A solution of APT3068 in water (37.5 mM, 21.3 uL) was added to a solution of TCEP (tris-2-carboxyethyl phosphine) (1 equiv.) in HEPES buffer (pH 8.5, 40 mM, 265 uL). A solution of APT2501 (1 mg/mL, 140 uL) was then added. The reaction mixture was left at ambient temperature overnight and then analysed by HPLC. The ligated product eluted slightly later than APT2501 and was found to be a 1/1 ligation product by ESMS-2444 (M+H). This product was purified by preparative HPLC and lyophilised. This product was a mixture of two products with single ligation to one or other of the 1-amino-2-thiols. These products are functionally equivalent and have identical molecular weight. FIG. 5 shows only one of these two products for the sake of brevity.

EGFP MESNA thioester lyophilisate was dissolved in HEPES buffer (pH 8.5 40 mM, 250 uL) and added to a solution of TCEP (tris-2-carboxyethyl phosphine) (1 equiv.) in HEPES buffer (pH 8.5, 40 mM, 265 uL). A solution of the singly ligated product described above in the same buffer (25 fold molar excess) was then added and the mixture incubated overnight at ambient temperature. SDS PAGE showed clear and quantitative conversion of the protein thioester to a ligated product approximately 2 kDa higher in molecular weight.

### Example 7

### Formation of a Trimerisation Reagent (APT3288)

Standard Fmoc solid phase synthesis was utilised on Rink amide resin. Monomer was coupled first followed by Fmoc-6-aminocaproic acid (twice) followed by Monomer. Two further additions of Fmoc-6-aminocaproic acid and one of Boc-Cys(Trt)-OH completed the synthesis. After cleavage from the resin and removal of all protecting groups using TFA/EDT/TIPS (95/5/5) the crude linker was ether precipitated and purified by preparative C18-reverse phase HPLC. ESMS 1035.4 (M+H).

1. A compound comprising a polypeptide, the polypeptide having at a C-terminal end a pseudo amino acid, wherein the pseudo amino acid has a side chain containing a 1-amino-2-thiol moiety.

2. A compound according to claim 1, wherein the polypeptide comprises the structure: 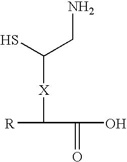
wherein:
R is a polypeptide chain;
X is a linker.

3. A compound according to claim 2, wherein the linker comprises (CH₂)n, wherein n is 0 to 6.

4. A protected pseudo amino acid comprising the structure 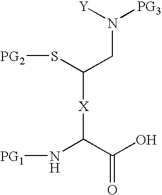
wherein:
Y is optionally H or other suitable residue; and
PG₁, PG₂ and PG₃ are different protecting groups.

5. A protected pseudo amino acid according to claim 4, wherein the protecting groups are selected from the listed consisting of FMOC, BOC or Trt.

6. A protected pseudo amino acid according to claim 5 having the structure:

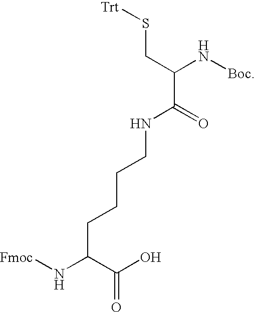

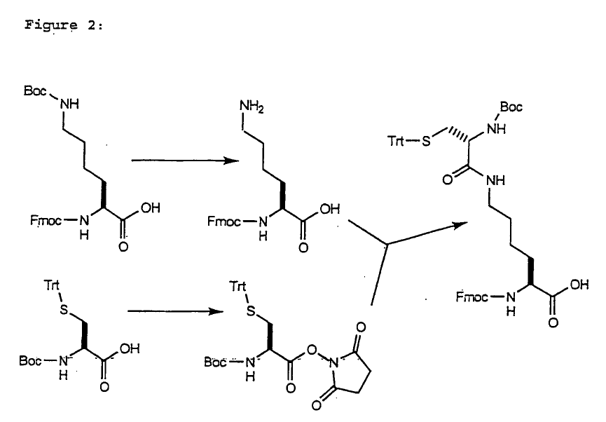

7. A process of producing a pseudo cysteine comprising the steps according to FIG. 2.

8. A pseudo cysteine obtained by the method of claim 7.

9. A pseudo cysteine obtainable by the method of claim 7.

10. The use of a polypeptide of claim 1 for producing a peptide, a protein by native chemical ligation, a homodimer, a heterodimer, or an oligomer.

11. The use of a pseudo amino acid of claim 4 for producing a peptide, a protein by native chemical ligation, a homodimer, a heterodimer, or an oligomer.

12. The use of a pseudo cysteine of claim 7 for producing a peptide, a protein by native chemical ligation, a homodimer, a heterodimer, or an oligomer.

13. (canceled)

14. A compound according to claim 3, wherein the n is 4.
